(19) United States
(12) Patent Application Publication
(10) Pub. No.: US 2006/0005742 A1
(43) Pub. Date: Jan. 12, 2006

(54) Pearl essence-analogous pearlescent pigments prepared by enzymatic reaction

(76) Inventors: Gernot Moeschl, Weiterstadt (DE); Doreen Warthe, Griesheim (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/530,874

(22) PCT Filed: Sep. 18, 2003
(86) PCT No.: PCT/EP03/10399

§ 371 Date: Apr. 11, 2005

(30) Foreign Application Priority Data
Oct. 9, 2002 (DE) 102 46 965.2

(51) Int. Cl. C09D 11/00; C08K 5/00; C07H 19/04; C12P 19/34

(52) U.S. Cl. 106/31.6; 106/499; 435/91.1; 544/243; 544/244; 536/26.1

(57) ABSTRACT

The present invention relates to pearl essence-analogous pearlescent pigments prepared by enzymatic reaction of nucleosides and/or nucleotides, to processes for the preparation thereof, and to the use of the pearl essence-analogous pearlescent pigments.

The present invention relates to pearl essence-analogous pearlescent pigments prepared by enzymatic reaction of nucleosides and/or nucleotides, to processes for the preparation thereof, and to the use of the pearl essence-analogous pearlescent pigments.

The first pearlescent pigment was natural pearl essence, which is obtained from fish scales and consists of the two purines guanine (75-97%) and hypoxanthine (3-25%). The most productive raw material sources are the scales of herring, bleak and sardine. The two-dimensional pigment crystals are extracted from aqueous dispersions of fish scales with organic solvents. In the process, the proteins are dissolved out, and the crystals are separated from the scales. The guanine and hypoxanthine crystals are isolated by centrifugation. In order to remove soluble proteins and fish oils, the flake-form crystals are finally washed.

Natural pearl essence preferentially crystallises as needles or oblong or hexagonal platelets which have a width of 1 to 10 μm, a length of 20 to 50 μm and a thickness of only 25 to 75 nm. Furthermore, pyramidal crystals occur (edge length about 10-30 μm). The optically anisotropic crystals have a refractive index of about 1.8 and a comparatively low density of 1.6 g/cm³.

Since the fish scales contain less than 1% of the pigment and in addition isolation is very complex, the prices of natural pearl essence are comparatively high. Furthermore, quality variations occur, making it impossible to use natural pearl essence as starting material in modern industry. The relatively high content of impurities results in yellowing and the evolution of a fish odour during storage. In addition, the pigment in the dry state is mechanically very sensitive and tends towards agglomeration. It is therefore only commercially available in the form of suspensions, usually having a solids content of 10 to 12%, under the name “pearl essence” or “essence d'orient”.

The low settling tendency obtained as a consequence of the low density and the silky and pearl-like lustre which occurs on addition of only small amounts are the reason why pearl essence has to date been able to defend its position in cosmetic formulations in spite of the above-mentioned disadvantages. The main area of use is in nail varnishes owing to the low sedimentation tendency and since the nitrocellulose lacquer used therein masks the fish odour.

There have been a few attempts to deposit guanine or other nitrogen-containing heterocyclic compounds in the form of flake-form crystals in order to prepare pearl essence-analogous pigments. Control of the crystallisation in the direction of flake-form crystals is vital in order to achieve a lustre effect, but has hitherto only been achieved with difficulty.

DE 1 242 778 describes a process for the conversion of volatile substances, such as, for example, guanine, into flakes by means of vacuum sublimation. This process is very complex and, owing to the requirements regarding apparatus, only suitable for the preparation of small amounts of pigment.

DE 1930265 discloses a process for the preparation of synthetic nacreous pigments in which nuclein bases, nucleosides or nucleotides are crystallised from solution. ⅕ to ¾ of the nuclein compound present in the aqueous solution is crystallised out over the course of 30 minutes, and the resultant crystals are allowed to mature for 1 to 24 hours. The crystallisation can be controlled via a number of parameters, such as pH, temperature or type of solvent. This process can only be reproduced poorly owing to this multiplicity of parameters. In addition, the solubility of the nuclein compounds is problematic. Thus, the nuclein bases, owing to their amphoteric character, are only soluble in strong acids or bases as corresponding salts. The solubility increases considerably at extreme pH values (around pH 0 or pH 14) and is at its lowest in the neutral region (pH 4.5-8). During dissolution of the crystals by modifying the pH, the respective ions become effective and are also incorporated into the crystal lattice. The crystallised salts are strongly basic or acidic on dissolution. On neutralisation, removal of the (alkali metal) cations or acid anions from the crystal lattice results in the formation of irregular precipitations of “shrunk structure” with no surface lustre.

The object was therefore to provide pearl essence-analogous pearlescent pigments which do not have the above-mentioned disadvantages of natural pearl essence, but have comparable lustre and a comparable appearance to natural pearl essence. In addition, the object was to provide a simple process for the preparation of pearl essence-analogous pearlescent pigments in good yields and with high purity and stability.

Surprisingly, it has been found that the pearl essence-analogous pearlescent pigments according to the invention and the process according to the invention for the preparation of these pearl essence-analogous pearlescent pigments meet the complex requirement profile outlined at the outset.

The present invention therefore relates to pearl essence-analogous pearlescent pigments prepared by enzymatic reaction of nucleosides and/or nucleotides with formation of the nucleobases in the form of flake-form crystals.

The present invention furthermore relates to a process for the preparation of pearl essence-analogous pearlescent pigments, characterised in that nucleosides and/or nucleotides are reacted enzymatically in one or more steps with formation of the nucleobases in the form of flake-form crystals. The pearl essence-analogous pearlescent pigments according to the invention are distinguished by a nature-identical lustre, good surface smoothness, high refractive index and high mechanical stability. Yellowing or the evolution of a fish odour do not occur in the case of the pearl essence-analogous pearlescent pigments according to the invention. The process according to the invention offers simple access to these pearl essence-analogous pearlescent pigments. Due to the use of enzymes, the reactions all proceed in the vicinity of the neutral point, meaning that no interfering cations or anions are incorporated into the crystal lattice. Furthermore, the use of enzymatic reactions results in even build-up of the flake-form crystals, resulting in pigments of high quality and uniformity.

The nucleosides or nucleotides employed in the process according to the invention can contain all pyrimidine and/or purine bases known to the person skilled in the art and further heterocyclic compounds of this type, such as, for example, guanine, hypoxanthine, adenine, cytosine, uracil and/or thymine. Preference is given to the use of purine base-containing nucleosides or nucleotides, but in particular guanine- and/or hypoxanthine-containing nucleosides or nucleotides.

In the process according to the invention, the enzymatic reaction is carried out by means of a hydrolase, a transferase (int. enzyme classification 2.4, 3.1 and 3.2) or by means of a fish scale extract. Preference is given to the use of naringinase, hesperidinase, nucleotidase, β-amylase, nucleosidase, inosinase and NAD-ase, in addition pentosyl transferases, in particular pyrophosphate phosphoribosyl transferases and purine nucleoside phosphorylase, and further enzymes which act in a similar manner and are known to the person skilled in the art, alone or in the form of a mixture. The enzymes can be obtained by all methods known to the person skilled in the art, for example by microbiological, chemical or extractive methods. Thus, besides the individual enzymes generally prepared by microbiological methods, a fish scale extract comprising the above-mentioned enzymes in addition to other constituents is also suitable for carrying out the process according to the invention. This extract is obtained from the fish scales and fish skin by aqueous extraction at neutral pH and subsequently freeze-dried. Besides other protein components, the resultant powder comprises the enzymes which can be employed in the process according to the invention. The enzymes or fish scale extract can be added in any desired form, preferably in aqueous solution.

When the process according to the invention is carried out, the nucleosides/nucleotides are dissolved in water with stirring. The pH of the solution is adjusted to a range of 3.5 to 8.0. The preferred pH is in the range from 4.4 to 7.0. Optimisation of the pH of the reaction mixture can be carried out in a manner which is obvious to the person skilled in the art.

The pH of the solution is adjusted by addition of buffer solutions. Buffer solutions which can be employed are all buffers known to the person skilled in the art, such as, for example, acetate, citrate or phosphate buffers. Preference is given to the use of citrate buffers, in particular sodium citrate buffer.

The temperature of the solution is adjusted to a range of 20 to 75° C. Preferred temperature ranges are 30 to 60° C. and can be matched to the optimum working range of the enzymes employed in a manner which is obvious to the person skilled in the art.

The enzymes or fish scale extract employed in the process according to the invention are added to the nucleoside/nucleotide solution prepared in this way. The enzymes enzymes or fish scale extract are or is preferably added in solution, in particular in aqueous solution. In a particular embodiment, the enzyme-containing solution can be stabilised by means of conventional additive concentrations of preservatives. All conventional preservatives can be employed, such as, for example, sorbic acid and derivatives thereof, the preservation preferably being carried out with sorbic acid.

The amount of enzyme employed depends on the desired rate of formation of the pearl essence-analogous pearlescent pigments taking into account the turnover ratio of the enzyme employed. It can be matched to the particular requirements by simple optimisation. In a preferred embodiment, the enzyme is added with decreasing number of enzyme units in a plurality of steps over the course of the reaction. The reaction duration is between 5 and 120 hours, preferably 12 to 72 hours and in particular 12 to 36 hours.

In order to achieve improved crystallisation of the pearl essence-analogous pearlescent pigments, pearl essence already prepared in this way or natural pearl essence can be added in order to inoculate the reaction solution.

Furthermore, surface-active substances can be added to the reaction mixture in the process according to the invention in order to control the surface and crystal structure of the pearl essence-analogous pearlescent pigments. To this end, use can be made of all nonionic and/or ionic surfactants known to the person skilled in the art, as described, for example, in K. Lindner, Tenside, Textilhilfsmittel, Waschrohstoffe [Surfactants, Textile Auxiliaries, Detergent Raw Materials], Vol. I, Wissenschaftl. Verlags GmbH, Stuttgart 1964, and available from, inter alia, ICI. Preference is given to the use of polyalkylene glycols and polysorbates, such as, for example, nonylphenol polyglycol ether, sorbitan monolaurates (ethoxylated) or relatively long-chain alkylates, such as, for example, myristates, palmitates or stearates.

In a further embodiment of the process according to the invention, gel-forming polymeric substances can be employed in order to support the two-dimensional crystal formation and in order to produce high surface smoothness. To this end, use can be made of all gel formers known to the person skilled in the art, such as, for example, alginates, gelatine and gelatine derivatives, cellulose and cellulose derivatives, polyoses or starch and starch derivatives. Preference is given to the use of alginates, gum arabic, gelatine and gelatine derivatives and in particular xanthan gum.

In addition, colorants which results in coloration of the pearl essence-analogous pearlescent pigments can be added to the reaction mixture. Suitable colorants are all organic colorants known to the person skilled in the art, such as, for example, natural or synthetic dyes or pigments. Preference is given to the use of water-soluble, natural dyes, such as, for example, carmine or chlorophyllin.

The crystals can be isolated by all methods known to the person skilled in the art. The crystals are preferably isolated by centrifugation of the reaction mixture one or more times followed by washing of the crystals, preferably with water. For further use, the pearl essence-analogous pearlescent pigments can be dried.

The pearl essence-analogous pearlescent pigments obtained by the process according to the invention exhibit a lustre which is comparable with natural pearl essence at the same time as improved accessibility. The disadvantages which arise in the case of natural pearl essence, such as a yellowish cast or fish odour, do not arise in the case of the pearlescent pigments according to the invention. Furthermore, the pearl essence-analogous pearlescent pigments prepared in accordance with the invention can be employed and used in dry form, which, besides use in the cosmetics established for pearl essence, also opens up other areas of application, such as, for example, industrial applications. In addition, the pearl essence-analogous pearlescent pigments according to the invention, in a similar manner to natural pearl essence, prove to be more mechanically stable than other inorganic, flake-form effect pigments, for example based on oxide. This results in an improvement in the potential for incorporation compared with inorganic effect pigments. In addition, the amount of pearl essence-analogous pearlescent pigments according to the invention which is necessary in order to achieve a pearlescent effect is smaller than in the case of oxide- or metal-based pigments.

The present invention thus also relates to the use of the pearl essence-analogous pearlescent pigments according to the invention in paints, coatings, printing inks, plastics, cosmetic formulations, in the foods sector, in paper, in security materials, in agricultural sheeting, for colouring seed, for colouring foods or in medicament coatings, in dry preparations or in pigment preparations.

In the case of cosmetic formulations, the pigments according to the invention are particularly suitable for decorative cosmetic products, such as, for example, nail varnishes, colouring powders, lipsticks or eye shadow. Analogous examples of the use of inorganic effect pigments in cosmetic formulations and materials which are suitable for this purpose are described in Research Disclosure, RD 471001. The effect pigments described therein can advantageously be replaced by the pearl essence-analogous pearlescent pigments according to the invention.

On use of the pigments in paints and coatings, all areas of application known to the person skilled in the art are possible, such as, for example, powder coatings, automobile finishes, printing inks for gravure, offset, screen or flexographic printing and for coatings in outdoor applications. In addition, the pigments according to the invention can be used for the pigmentation of films and plastics, for example for agricultural sheeting, gift foils, plastic containers and mouldings for all applications known to the person skilled in the art. In addition, the pigments according to the invention are also suitable for use in security printing and in security-relevant features for, for example, counterfeiting-proof cards and identity papers, such as, for example, entry tickets, personal identity cards, banknotes, cheques and cheque cards and for other counterfeiting-proof documents.

In addition, the pigments according to the invention are suitable for the production of pharmaceutical products, such as, for example, tablet coatings or as powder additive and in the foods sector for colouring foods. The effect pigments according to the invention are furthermore suitable for the preparation of pigment preparations, such as, for example, pearlets, pastes and suspensions and of dry preparations, such as, for example, pellets, granules, chips, etc., which are preferably used in printing inks and coatings. The pigments according to the invention can be employed in a multiplicity of known binders used in colour systems and can be used in both aqueous and solvent-based systems. The pigments can be incorporated into the respective application media by all methods known to the person skilled in the art.

In addition, the effect pigments according to the invention can also advantageously be mixed with all known organic and/or inorganic colorants, such as, for example, organic dyes, organic pigments, inorganic single-layered or multilayered pigments, inorganic dyes or pigments.

The invention accordingly also relates to paints, coatings, printing inks, plastics, cosmetic formulations, food products, paper, security materials, agricultural sheeting, seed, foods or medicaments, dry preparations or pigment preparations comprising the pearl essence-analogous pearlescent pigments according to the invention and the pearl essence-analogous pearlescent pigments prepared by the process according to the invention.

The following examples are intended to explain the invention in greater detail, but without limiting it.

### EXAMPLES

### Example 1

1.0 g of guanosine monophosphate sodium salt heptahydrate (E 627) and 0.25 g of inosine monophosphate sodium salt (E 631) are dissolved in 150 ml of demin. water, and 250 mg of magnesium sulfate and 2.5 g of tetrasodium diphosphate and 20 mg of nonylphenol polyglycol ether are added. The mixture is inoculated with a little pearl essence, for example 10 to 20 mg, in order to accelerate crystal formation. The slightly basic solution is adjusted to a pH of 7.2 using 10% citric acid, and the volume of the solution is made up to 200 ml. About 20 U of purine pyrophosphate phosphoribotransferase (2.4.2.8.), dissolved in a little water, are then added. The solution is warmed to 40° C. and slowly stirred in a closed apparatus. In the first 10 hours, 8 U of the enzyme per hour are metered in continuously. In the other 48 hours, the metering is reduced in steps to 4 U and later to 2 U. The enzyme solution is preserved with 0.05% sorbic acid. Highly lustrous, very thin crystal platelets of various size, with a length in the order of 5-30 μm and a width in the order of 2-12 μm, are formed. The virtually water-insoluble fine laminates are centrifuged off, washed three times with demin. water and dried under gentle conditions (amount produced about 320 mg).

### Example 2

As Example 1, using purine nucleoside phosphorylase (2.4.2.1)

### Example 3

7.5 g of guanosine monophosphate Na salt and 2.5 g of inosine/guanosine monophosphate Na salt (1:1 mixture) are dissolved in 750 ml of demin. water. 250 ml of an Na citrate buffer solution having a pH of 4.4 are added (conc. of citrate about 6%). The enzymes naringinase (amount/activity about 100 U) and hesperidinase (amount/activity about 200 U) dissolved in a little water are added. For better crystal growth, about 200 mg of riboside are added. The solution is stirred slowly at 44° C. for about 24 hours in a closed reaction vessel. After the first 15 hrs, half the amount (50 U and 100 U) of the above-mentioned enzymes is again added.

Needle- and laminate-like crystals having a high pearlescent effect are formed and are centrifuged off three times and washed with demin. water. They are sparingly soluble in water and virtually insoluble in organic solvents and can be employed in this form as pearlescent pigments.

### Example 4

4 g of riboside compounds from Example 3, calculated on the basis of the dry weight, are dispersed in 300 ml of demin. water and adjusted to a pH of 6.4 using 100 ml of citrate buffer solution (containing about 6% of citrate) and warmed to 54° C. with slow stirring. 0.02% of sorbitan monolaurate (ethoxylated) and 0.1% of xanthan gum, calculated on the basis of the approximately 400 ml of the batch, are added. 150 U of β-amylase (3.2.1.2.) are employed as initial activity. In the subsequent 48-72 hours, initially 15 U, later 10 U or 5 U of enzyme activity are metered in per hour. The enzyme solution is preserved with 0.05% sorbic acid. The suspension is inoculated with about 40 mg of fine pearl essence flakes.

Rectangular, highly lustrous and high-refractive-index pearlescent pigment flakes with widely varying sheet-like dimensions, for example with a width of 3-20 μm to a length of 5-50 μm, are formed. The very thin, laminate-like flakes, whose mass yield is greater than 50%, are centrifuged, washed and dried under gentle conditions. Alternatively, for example, it is possible for acetone to be added to them in the final washing process in order to remove adherent water, and for them then to be transferred directly into, for example, cellulose nitrate or, for example, castor oil. These formulations can be employed directly for applications, for example, in nail varnishes or lip cosmetics.

### Example 5

4 g of the washed riboside compounds from Example 4 are dispersed in 400 ml of demin. water and adjusted to a pH of 5.4 using 100 ml of citrate buffer solution (containing about 6% of citric acid). Initially about 100 U of nucleosidase (3.2.2.1.) are metered in. The remainder of the enzyme activities are then metered in very slowly from a preserved, diluted enzyme solution, as in Example 4. Within about 72 hours, the metering rate is reduced from about 10 U to 2 U/hour. The suspension is inoculated with about 40 mg of pearl essence. Highly lustrous, high-refractive-index pigment flakes which are very similar to pearl essence obtained from herring or trout are formed. They are predominantly oblong laminates rounded at the end with a width of 1-5 μm and a length of 3 to 12 μm. They can be isolated and processed further analogously to Example 4.

### Example 6

### Nail Varnish

98.50% by weight of thixotropic nail varnish base 1348 (International Laquers S.A., comprising toluene, ethyl acetate, butyl acetate, nitrocellulose, tosylamide-formaldehyde resin, dibutyl phthalate, isopropanol, stear-alkonium hectorite, camphor, acrylate copolymer, benzophenone) and 1.5% by weight of a dispersion as described in Example 4 (11% by weight of pearl essence-analogous pearlescent pigments in castor oil) are weighed out together, mixed by hand using a spatula and subsequently stirred at 1000 rpm for 10 min. A nail varnish having uniform pearlescence is obtained.

### Example 7

### Lipstick

Phase A

| Raw material | % by wt. |
|---|---|
| Castor oil | 63.85 |
| Beeswax | 12.50 |
| Isopropyl myristate | 8.00 |
| Carnauba wax | 7.50 |
| Lanolin | 5.00 |
| Paraffin, viscous | 3.00 |
| Propylparaben | 0.10 |
| Oxynex ® 2004 (antioxidant from Merck KGaA) | 0.05 |
| | 100.00 |

Phase B

Dispersion as described in Example 4 comprising 11% by weight of pearl essence-analogous pearlescent pigments in castor oil.

Preparation

Phase A is heated to 80° C. and mixed well, phase B is warmed to 60° C. 80% by weight of phase A and 20% by weight of phase B are subsequently mixed in a casting apparatus, and the temperature is adjusted to about 60° C. The homogeneous melt is poured into casting moulds pre-warmed to 60° C. and allowed to cool. The castings are removed from the moulds when cold and, after warming to room temperature, treated briefly with a flame. The sticks produce uniform pearlescence on the skin.

### Example 8

### Coating

A commercially available acrylate/melamine-based basecoat, dissolved in a butyl acetate/xylene mixture comprising 1.5% by weight of the pigment from Example 1, is applied to a pale-primed metal sheet by compressed air spraying. After a drying time of 60 seconds, a varnish based on acrylate which is hardened with isocyanate is applied.

A lustrous coating with good hiding power is obtained.

1. Pearl essence-analogous pearlescent pigments prepared by enzymatic reaction of nucleosides and/or nucleotides with formation of the nucleobases in the form of flake-form crystals.

2. Process for the preparation of pearl essence-analogous pearlescent pigments according to claim 1, characterised in that nucleosides and/or nucleotides are reacted enzymatically in one or more steps with formation of the nucleobases in the form of flake-form crystals.

3. Process according to claim 2, characterised in that the nucleoside and/or nucleotide is a pyrimidine and/or purine nucleoside/nucleotide.

4. Process according to claim 2, characterised in that the nucleoside and/or nucleotide comprises guanine and/or hypoxanthine.

5. Process according to claim 2, characterised in that the enzymatic reaction is carried out by means of a hydrolase, a transferase or a fish scale extract.

6. Process according to claim 2, characterised in that the enzymatic reaction is carried out with the enzymes naringinase, hesperidinase, nucleotidase, β-amylase, nucleosidase, inosinase, NAD-ase, pyrophosphate phosphoribosyl transferase, purine nucleoside phosphorylase or with a mixture thereof.

7. Process according to claim 2, characterised in that the enzyme or enzyme mixture is metered in with decreasing enzyme units in a plurality of steps.

8. Process according to claim 2, characterised in that surface-active substances are additionally added.

9. Process according to claim 2, characterised in that gel-forming polymeric substances are additionally added.

10. Process according to claim 2, characterised in that dyes are additionally added.

11. Use of pearl essence-analogous pearlescent pigments according to claim 1 in paints, coatings, printing inks, plastics, cosmetic formulations, in the foods sector, in paper, in security materials, in agricultural sheeting, for colouring seed, for colouring foods or in medicament coatings, in dry preparations or in pigment preparations.

12. Paints, coatings, printing inks, plastics, cosmetic formulations, food products, paper, security materials, agricultural sheeting, seed, foods or medicaments, dry preparations or pigment preparations comprising pearl essence-analogous pearlescent pigments according to claim 1.
